(19) United States
(12) Patent Application Publication
(10) Pub. No.: US 2020/0239562 A1
(43) Pub. Date: Jul. 30, 2020

(54) ANTI-IL-33 THERAPY FOR ATOPIC DERMATITIS

(71) Applicant: AnaptysBio, Inc., San Diego, CA (US)

(72) Inventor: Marco LONDEI, San Diego, CA (US)

(73) Assignee: AnaptysBio, Inc., San Diego, CA (US)

(21) Appl. No.: 16/755,002

(22) PCT Filed: Oct. 9, 2018
(86) PCT No.: PCT/US2018/054989

§ 371 Date: Apr. 9, 2020

Related U.S. Application Data
(60) Provisional application No. 62/569,994, filed on Oct. 9, 2017.

(51) Int. Cl. C07K 16/24; A61K 9/00; A61P 17/04

(52) U.S. Cl. CPC C07K 16/244; A61K 2039/545; A61P 17/04; A61K 9/0019

(57) ABSTRACT

Methods of treating atopic dermatitis in a patient with an anti-IL-33 antibody, and methods of selecting atopic dermatitis patients for anti-IL-33 therapy.

### CROSS-REFERENCE TO RELATED APPLICATION

This patent application claims the benefit of U.S. Provisional Patent Application No. 62/569,994 filed Oct. 9, 2017, which is incorporated herein by reference in its entirety.

### INCORPORATION BY REFERENCE OF MATERIAL SUBMITTED ELECTRONICALLY

Incorporated by reference in its entirety herein is a computer-readable nucleotide/amino acid sequence listing submitted concurrently herewith and identified as follows: One 3,917 Byte ASCII (Text) file named “740834_ST25.txt,” created on Oct. 6, 2018.

### BACKGROUND OF THE INVENTION

Atopic dermatitis (AD) is a common chronic inflammatory disease, characterized by pruritic skin lesions, which affects a significant percentage (up to 10%) of the adult population in the United States. An increasing body of evidence has linked AD to other allergic diseases such as asthma and food allergies. AD is also part of a process called the atopic march, a progression from AD to allergic rhinitis and asthma. Despite the clear clinical and epidemiological link between these diseases, treatments for AD have been lagging as compared to asthma and other allergic diseases.

AD represents an increasing socio-economic burden as a significant and increasing proportion of individuals suffer from AD. Moreover, the true impact of the chronic clinical manifestation of AD, including the unmet medical need caused by AD, has only begun to be acknowledged. This historical lack of recognition of the importance of the unmet medical need is underscored by the delay in testing biologics in these patients.

Historically, the standard of care for AD patients has been centered on the use of topical drugs (i.e. corticosteroids) to locally control the manifestations of the disease on the skin. Recently biologics (i.e. monoclonal antibodies, “Mabs”) have been tested for AD, few of which have delivered promising results. One Mab tested which provided results is Dupilumab (Dupixent®), which was recently approved for use in moderate to severe AD patients. Dupilumab (Dupixent®) targets the IL-4Ra molecule which is functionally shared by the IL-4 and IL-13 receptor, thus inhibiting both IL-4 and IL-13. However, Dupilumab (Dupixent®) suffers from certain intrinsic pharmacokinetic limitations which hampers its widespread use. For example, Dupilumab (Dupixent®) has a very short half-life which imposes a frequent dosing schedule of biweekly or weekly administration of large dosages, i.e., up to 300 mg per dose of antibody. Another monoclonal antibody, Mepolizumab (Nucala®), which targets IL-5, has been approved for certain forms of asthma but have failed to provide significant benefit to AD patients. Therefore, there is an unmet need for an effective biologic with a long functional pharmacological activity for patients with AD.

### BRIEF SUMMARY OF THE INVENTION

In an embodiment, the invention provides a method of treating atopic dermatitis in a patient comprising administering to the patient a dose of an anti-IL-33 antibody or antigen binding fragment thereof not more than once every two weeks.

In another embodiment, the invention provides a method of treating atopic dermatitis where a dose of anti-IL-33 antibody or antigen binding fragment thereof results in greater than 50% reduction in the EASI score for a patient.

In another embodiment, the invention provides a method of selecting a patient with atopic dermatitis for treatment with an anti-IL-33 antibody or antigen binding fragment thereof, the method comprising (a) administering an anti-IL-33 antibody or antigen binding fragment thereof to the patient, and (b) comparing the level of white blood cells in a disease lesion on the patient's skin after administration of the anti-IL-33 antibody or antigen binding fragment thereof to the level of white blood cells in a disease lesion on the patient's skin before administration of the anti-IL-33 antibody or antigen binding fragment thereof, wherein the patient is selected for treatment when a decrease in the population of eosinophils, basophils, monocytes or neutrophils is observed after administration of the anti-IL-33 antibody or antigen binding fragment thereof.

In another embodiment, the invention provides a method of selecting a patient with atopic dermatitis for treatment with an anti-IL-33 antibody or antigen binding fragment thereof, the method comprising (a) administering an anti-IL-33 antibody or antigen binding fragment thereof to the patient, and (b) comparing the level of itching or pruritus exhibited by the patient after administration of the anti-IL-33 antibody or antigen binding fragment thereof to the level of itching or pruritus exhibited by the patient before administration of the anti-IL-33 antibody or antigen binding fragment thereof.

In additional embodiments, the invention provides anti-IL-33 antibodies and antigen binding fragments suitable for use in the inventive method.

### BRIEF DESCRIPTION OF THE SEVERAL VIEWS OF THE DRAWING(S)

FIG. 1 is a schematic of the study design according to embodiments of the invention.

FIGS. 2A-2E are graphs showing severity scores after administration of placebo on day 1 and 300 mg IV ANB020 on day 8, according to embodiments of the invention.

FIG. 2A is a graph showing the percentage change in EAI score based on changes from day 1. The Y axis is the percent reduction in EASI score. The X axis is the number of days. Friedman test with Dunn's multiple comparison. n=12, showing mean+/−SD. *P<0.05, **P<0.01, ***P<0.001.

FIG. 2B is a graph showing the percentage of patients reaching EASI50 and EASI75. The Y axis is the percentage of patients. The X axis is the number of days. Friedman test with Dunn's multiple comparison. n=12, showing mean+/−SD. *P<0.05, **P<0.01, ***P<0.001.

FIG. 2C is a graph showing changes in absolute EASI score. The Y axis is the absolute EASI score. The X axis is the number of days. Friedman test with Dunn's multiple comparison. n=12, showing mean+/−SD. *P<0.05, **P<0.01, ***P<0.001.

FIG. 2D is a graph showing changes in absolute SCORAD score. The Y axis is the absolute SCORAD score. The X axis is the number of days. Friedman test with Dunn's multiple comparison. n=12, showing mean+/−SD. *P<0.05, **P<0.01, ***P<0.001.

FIG. 2E is a graph showing changes in IGA absolute score. The Y axis is the absolute IGA score. The X axis is the number of days. Friedman test with Dunn's multiple comparison. n=12, showing mean+/−SD. *P<0.05, **P<0.01, ***P<0.001.

FIGS. 3A-3B are graphs showing patient reported outcome measures after administration of placebo on day 1 and 300 mg IV ANB020 on day 8, according to embodiments of the invention.

FIG. 3A is a graph showing percentage changes in DLQI score. The Y axis is the percent change in DLQI. The X axis is the number of days. Friedman test with Dunn's multiple comparison. n=12, showing mean+/−SD. *P<0.05, **P<0.01, ***P<0.001.

FIG. 3B is a graph showing percentage changes in 5D itch score. The Y axis is the percent change in the 5D itch score. The X axis is the number of days. Friedman test with Dunn's multiple comparison. n=12, showing mean+/−SD. *P<0.05, **P<0.01, ***P<0.001.

FIGS. 4A-4B are graphs showing pharmacodynamic effects after administration of placebo on day 1 and 300 mg IV ANB020 on day 8, according to embodiments of the invention.

FIG. 4A is a graph showing the percentage of IFNγ production over time. The Y axis is the percentage of IFNγ production. The X axis is the number of days. Friedman test with Dunn's multiple comparison. n=12, showing mean+/−SD.

FIG. 4B is a graph showing the correlation between IFNγ production in response to IL-33/IL-12 and EASI %. The Y axis is the IFNγ concentration in pg/ml. The X axis is the EASI score. **P<0.01, ***P<0.001.

FIGS. 5A-5D are graphs showing skin and blood biomarkers after administration of placebo on day 1 and 300 mg IV ANB020 on day 8, according to embodiments of the invention.

FIG. 5A is a graph showing absolute peripheral blood eosinophil count (in 10⁹/L). The Y axis is the absolute eosinophil count. The X axis is the number of days. n=12, non-parametric t test. *P=0.05.

FIG. 5B is a graph showing the correlation between eosinophil percentage and EASI score. The Y axis is the percentage of eosinophils. The X axis is the EASI score. n=12, non-parametric t test. *P=0.05.

FIG. 5C is a graph showing the results of the House Dust Mite (HDM) challenge. The Y axis is the percentage of skin granulocytes. The X axis plots each patient at post-placebo and post ANB020 administration. Granulocytes were quantified using flow cytometry and expressed as percentage total leucocytes. n=12, non-parametric t test. *P=0.05.

FIG. 5D is a graph showing additional results of the House Dust Mite (HDM) challenge. The Y axis is the percentage of skin granulocytes. The X axis plots each patient at post-placebo and post ANB020 administration. Granulocytes were quantified using flow cytometry and expressed as percentage total leucocytes. n=12, non-parametric t test. *P=0.05.

### DETAILED DESCRIPTION OF THE INVENTION

Interleukin-33 (hereinafter referred to as IL-33) is a cytokine of the interleukin-1 family, which is involved in inflammatory conditions. IL-33 is constitutively expressed in the nuclei of epithelial cells and vascular endothelial cells, is released during cell destruction following tissue injury caused by infections or physical or chemical stress, and then acts as alarmin. The extracellularly released IL-33 binds to IL-33 receptors expressed on cells, thereby being capable of activating intracellular signal transduction. IL-33 receptors are expressed on various immune cells and epithelial cells, where IL-33-induced intracellular signal transduction occurs.

IL-33 is believed to induce allergic inflammation (for example, asthma, atopic dermatitis, pollinosis, and anaphylactic shock) by inducing production of Th2 cytokines (for example, IL-4, IL-5, IL-6, and IL-13) from Th2 cells, mast cells, eosinophils, basophils, natural killer T (NKT) cells, and Group 2 innate lymphocytes, among immune cells expressing IL-33 receptors (Ohno et al., Allergy, Vol. 67, p. 1203 (2012)). Increased IL-33 expression is observed in various human inflammatory diseases (for example, rheumatoid arthritis, asthma, systemic sclerosis, fibrosis such as hepatic fibrosis and pulmonary fibrosis, psoriasis, ulcerative colitis, Crohn's disease, multiple sclerosis, and ankylosing spondylitis), and IL-33 is believed to be involved in the development and maintenance of various diseases (see e.g., Matsuyama et al., J. Rheumatology, Vol. 37, p. 18 (2010); Prefontaine et al., J. Allergy Clin. Immunol., Vol. 125, p. 752 (2010); Yanaba et al., Clin. Rheumatol., Vol. 30, p. 825 (2011); and Rankin et al., J. Immunol., Vol. 184, p. 1526 (2010).

IL-33 is believed to be involved in initiation and progression of AD. Importantly IL-33 has been shown to be the essential molecule consolidating the function of pathogenic Th2 cells both in humans and rodents. IL-33 has been shown to drive Th2 responses in asthma and atopic dermatitis by acting on a series of white cells intrinsically involved in the pathogenesis of atopic disorders. Furthermore, IL-33 is involved in controlling the rapid release of downstream cytokines such as IL-5, IL-4, and IL-13. Additionally, genetic and functional studies have demonstrated the central role of IL-33 and its receptor ST2 in predisposing to the development of atopic dermatitis in patients and animal models.

In an embodiment, the invention provides a method of treating atopic dermatitis in a patient comprising administering to the patient a dose of an anti-IL-33 antibody or antigen binding fragment thereof not more than once every two weeks. As used herein, the term “dosing” refers to a single administration of a substance (i.e., IL-33 antibody or antigen binding fragment) to achieve a therapeutic objective. Dosage administration of not more than once every two weeks has many advantages over weekly dosing including, but not limited to, a lower number of total injections, decreased number of injection site reactions (e.g., local pain and swelling), increased patient compliance, and lower cost to patients and health care providers. Subcutaneous dosing is advantageous because the patient may self-administer a therapeutic substance, e.g., an anti-IL-33 antibody or antigen fragment thereof, which is convenient for both the patient and the health care provider.

The dosage administration regimen of the inventive method may be adjusted to provide the optimum desired response (e.g., treatment of the patient) and, in some embodiments, even less frequent dosing is required. Thus, in additional embodiments, the inventive method may comprise administering to the patient a dose of an anti-IL-33 antibody or antigen binding fragment thereof, for example, not more than once every three weeks, not more than once every four weeks, not more than once every six weeks, or not more than once every eight weeks. In still further embodiments, the method comprises administering to the patient a dose of an anti-IL-33 antibody or antigen binding fragment thereof not more than once every 10 weeks, not more than once every 12 weeks, not more than once every 16 weeks, or even not more than once every 20 weeks.

The individual dose of the invention may be a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may vary according to factors such as the disease state, age, sex, and weight of the patient, and the ability of the antibody or antibody portion to elicit a desired response in the individual. A therapeutically effective amount is also one in which any toxic or detrimental effects of the antibody or antibody portion are outweighed by the therapeutically beneficial effects. A “prophylactically effective amount” refers to an amount effective, at dosages and for periods of time necessary, to achieve the desired prophylactic result. The prophylactically effective amount may be less than the therapeutically effective amount.

An exemplary, non-limiting range for a therapeutically or prophylactically effective dose of an antibody or antibody binding fragment according to the invention is at least about 40 mg, such as at least about 50 mg, at least about 60 mg, at least about 70 mg, at least about 80 mg, at least about 90 mg, or at least about 100 mg. In some embodiments, the dose may be as at least about 200 mg, or at least about 300 mg. Typically, the dose will be less than about 1000 mg, such as less than about 800 mg, or less than about 700 mg (e.g., less than about 600 mg, less than about 500 mg, or less than about 400 mg). Any of the foregoing can also be expressed as ranges (e.g., about 40-1000 mg, 40-800 mg, 40-600 mg, 40-400 mg, 50-1000 mg, 50-800 mg, 50-600 mg, 50-400 mg, 60-1000 mg, 60-800 mg, 60-600 mg, 60-400 mg, 70-1000 mg, 70-800 mg, 70-600 mg, 70-400 mg, 80-1000 mg, 80-800 mg, 80-600 mg, 80-400 mg, 100-1000 mg, 100-800 mg, 100-600 mg, 100-400 mg, 200-1000 mg, 200-800 mg, 200-600 mg, 200-400 mg, 300-1000 mg, 300-800 mg, 300-600 mg, 300-400 mg, etc., including any sub-ranges thereof (e.g., about 250-350 mg, etc.). Thus, for instance, the single dose can be about 100 mg, 200 mg, 300 mg, 400 mg, 500 mg, 600 mg, 700 mg, 800 mg, 900 mg, or 1000 mg.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

In embodiments, the dosages of the invention may include a “loading dose” and a “maintenance dose” of an antibody or antibody portion of the invention, each of which is in an amount as described above. The loading dose may be higher than the maintenance dose, equivalent to the maintenance dose, or lower than the maintenance dose. In an embodiment, the loading dose is four times, three times, two times, one and one half times the maintenance dose, or equal to the maintenance dose.

Furthermore, the loading dose can be administered at any time before the first maintenance dose (e.g., at least 1, 2, 3, 5, 7, 10, or 14 days before; in some embodiments less than 3 weeks, less than 4 weeks, less than 8 weeks, or less than 12 weeks before). In an embodiment, the anti-IL-33 antibody or antigen binding fragment thereof is administered to a patient treatment according to a schedule, wherein a least one loading dose is first administered, and, second, at least one therapeutically effective maintenance dose is administered. In a further embodiment, the loading dose may be administered on day 1 and the maintenance dose may be administered not more than once every two weeks after administration of the loading dose. In additional embodiments, the loading dose may be administered on day 1, and the maintenance dose may be administered not more than once every three weeks, not more than once every four weeks, not more than once every six weeks, or not more than once every eight weeks (e.g., not more than once every twelve weeks, not more than once every sixteen weeks, or not more than once every 20 weeks) after administration of the loading dose.

In preferred embodiments, the method of treating atopic dermatitis has a rapid and persistent effect. This effect can be measured by any suitable metric, but one widely used measure is the Eczema Area and Severity Index (EASI), which an investigator-assessed instrument measuring the severity of clinical signs in AD. It ranges from 0 (no eczema) to 72. EASI is one of the core outcome instruments recommended to be included in all clinical trials on atopic dermatitis. Additional widely used metrics are the 5-D Itch (Pruritis) Scale (discussed below), SCORAD (“SCORing Atopic Dermatitis”), a clinical tool for assessing the severity (i.e., extent, intensity) of atopic dermatitis, DLQI (Dermatology and Life Quality Index, which is a questionnaire with 10 items used to measure the quality of life of dermatological patients, and the 5-point Investigator's Global Assessment (IGA) Scale which is a tool for evaluating plaque psoriasis severity in clinical trials. Each of these methods is known to a person of skill in the art.

In an embodiment, the method of the invention provides a therapeutic effect such that the patient achieves a 50% improvement relative to the patient's baseline EASI score within six weeks, preferably within four weeks, more preferably within 3 weeks or even within 2 weeks of beginning treatment. In another embodiment, the patient achieves a 50% improvement relative to the patient's baseline EASI score within six weeks, preferably within four weeks, more preferably within 3 weeks or even within 2 weeks of receiving a dose of the anti-IL-33 antibody, which dose can be in an amount as described herein. In a further embodiment, the method provides a therapeutic effect such that a population of at least 100 patients achieves a 50% improvement in at least 50% of the patients relative to their baseline EASI score within four weeks, within three weeks, or even within two weeks of beginning treatment; or within four weeks, within three weeks, or even within two weeks of a receiving a dose of the anti-IL-33 antibody, which dose is an amount as described herein.

In some embodiments, the method provides a persistent therapeutic effect such that the patient (or population of patients) maintains reduced blood eosinophil count over a significant period of time. Thus, in some embodiments, about 20 days or more or even longer (e.g, about 30 days or more, about 40 days or more, about 50 days or more, about 60 days or more, about 70 days or more, about 80 days or more, about 90 days or more, about 100 days or more, about 110 days or more, about 120 days or more, about 130 days or more, or even about 140 days or more) after administration of a dose (e.g., a single dose) of anti-IL-33 antibody, the patient has a reduction in the patient's baseline blood eosinophil count of at least 10%, at least 20%, at least 30%, at even least 40%, or the population of patients has an average reduction in the patient's baseline blood eosinophil count of at least 10%, at least 20%, at least 30%, at even least 40%.

In addition, or instead, the method provides a therapeutic effect such that the patient maintains a 50% improvement relative to the patient's baseline EASI score for at least 3 weeks, at least 4 weeks, at least 5 weeks, at least 6 weeks, at least 7 weeks, at least 8 weeks, at least 9 weeks, at least 10 weeks, at least 12 weeks, or even longer after administration of a dose of anti-IL-33 antibody.

### Methods for Selecting Suitable Patients for Treatment

Also provided herein is a method of selecting a patient with AD for treatment with an anti-IL-33 antibody, such as any method of treatment described herein. Methods for selecting suitable patients for treatment with the inventive methods may vary.

Pruritis, or itch, is the hallmark of atopic dermatitis, and has a significant impact on quality of life for patients with this disease. Various central and peripheral mediators have been suggested to play a role in the pathophysiology of atopic eczema itch. Significant cross-talk occurs among stratum corneum, keratinocytes, immune cells, and nerve fibers, which are in close proximity to one another and induce itch. The impaired barrier function associated with the itch-scratch cycle further augments this vicious cycle (Yosipovitch et al., Current Allergy Asthma Rep., 8(4), 306-311 (2008)). Without being bound to a particular theory or mechanism, it is believed that administration of the inventive anti-IL-33 antibody or antibody fragment thereof to a patient with atopic dermatitis may lead to an increase or decrease in the level of pruritus exhibited by the person after administration of the inventive anti-IL-33 antibody or antibody fragment thereof. The degree of pruritus experienced by a patient may be assessed by the 5-D Itch scale. The 5-D Itch scale is able to detect change over time, which is essential to determining the type and duration of pharmaceutical intervention and/or treatment. The 5-D itch scale was developed as a brief but multidimensional questionnaire designed to be useful as an outcome measure in clinical trials. The five dimensions are degree, duration, direction, disability and distribution (Elman et al., Br. J. Dermatol., 162(3): 587-593 (2010)).

In an embodiment, the invention provides a method of selecting a patient with atopic dermatitis for treatment with an anti-IL-33 antibody or antigen binding fragment thereof, the method comprising (a) administering an anti-IL-33 antibody or antigen binding fragment thereof to the patient; and (b) comparing the level of itching or pruritus exhibited by the patient after administration of the anti-IL-33 antibody or antigen binding fragment thereof to the level of itching or pruritus exhibited by the patient before administration of the anti-IL-33 antibody or antigen binding fragment thereof. Following the selection of a patient(s) with atopic dermatitis for treatment with an anti-IL-33 antibody or antigen binding fragment thereof by any of the selection methods described herein, the inventive method includes treating such patient with the inventive anti-IL-33 antibody or antigen binding fragment thereof.

As noted above, the adaptive and innate immune systems have important roles in the pathophysiology of AD. In AD pathogenesis, IL-33 is thought to be released by epithelial cells in various tissues and organs, including immune cells (i.e., white blood cells, or leukocytes) that initiate and orchestrate atopic responses. Without being bound by a particular theory or mechanism, it is believed that the anti-IL-33 antibody or antigen-binding fragment of the inventive method inhibits IL-33 function and acts upstream broadly across the key cell types involved in atopy. Such cells include different types of leukocytes which produce, transport, and distribute antibodies as part of the body's innate immune response. Granulocytes (so named because of granules in the cytoplasm) include neutrophils, eosinophils, and basophils. Nongranulocytes include lymphocytes and monocytes. Both granulocytes and nongranuloctyes are directly and indirectly involved in IL-33 function.

Analysis of the cellular and cytokine expression in AD skin lesions in on the skin of a patient presenting with atopic dermatitis, for example, with acute eczematous skin lesions that are intensely pruritic, may help determine whether such patient is a suitable candidate for treatment with antibodies or antibody binding fragments. Without being bound by a particular theory or mechanism, it is believed that the leukocytes involved in the pathogenesis of atopic dermatitis described above may be found in induced disease lesions, or blisters, formed in response to injection with a either a placebo or any form of allergen, including, for example, the house dust mite (a “skin challenge”). Such skin challenges may provide information helpful to clinicians in determining which patients may respond to treatment with an anti-IL-33 antibody or binding fragment thereof.

Without being bound by a particular theory or mechanism, it is believed that the population of eosinophils, basophils, monocytes or neutrophils may increase or decrease after administration of the inventive anti-IL-33 antibody or antigen binding fragment thereof to a patient with a condition susceptible to anti-IL-33 therapy. Thus, eosinophil reduction may be useful as a prognostic and treatment monitoring biomarker. Loss-of-function mutations in the IL-33 pathway have demonstrated reduced blood eosinophil levels (Smith et al., PLoS Genet. 13(3): e1006659 (2017)). The IL-33 receptor complex ST2/IL-1RAcP is expressed by many cells including Th2 cells, Tregs, ILC2, neutrophils, mast cells, eosinophils and basophils. When IL-33 is over-expressed in keratinocytes, this leads to an AD-like clinical phenotype with eosinophil, mast cell and ILC2 infiltration in to the skin, and IL-33 administration to the skin leads to skin infiltration by mast cells and neutrophils (Imai et al., Proc Natl Acad Sci U.S.A., 110(34): 13921-6 (2013); Hueber et al., Eur J Immunol. 41(8): 2229-37 (2011)).

In an embodiment, the inventive method provides a method of selecting a patient with atopic dermatitis for treatment with an anti-IL-33 antibody or antigen binding fragment thereof, the method comprising (a) administering an anti-IL-33 antibody or antigen binding fragment thereof to the patient; and (b) comparing the level of white blood cells (e.g., granulocytes, eosinophils, basophils, monocytes or neutrophils) in the patient's blood or in a disease lesion on the patient's skin after administration of the anti-IL-33 antibody or antigen binding fragment thereof to the level of white blood cells in the blood or a disease lesion on the patient's skin before administration of the anti-IL-33 antibody or antigen binding fragment thereof; wherein the patient is selected for treatment when a decrease in the population of white blood cells (e.g., granulocytes, eosinophils, basophils, monocytes or neutrophils) is observed after administration of the anti-IL-33 antibody or antigen binding fragment thereof. The foregoing method can be performed using a blood sample or sample from a skin lesion of the patient before and after the patient has been administered an anti-IL-33 agent. Thus, in a related aspect, there is provided a method of selection of a patient with atopic dermatitis for treatment with an anti-IL-33 antibody or antigen binding fragment thereof, the method comprising comparing the level of white blood cells in a blood sample or a sample from a disease lesion on the patient's skin before and after an anti-IL-33 antibody has been administered to the patient; and selecting the patient for treatment when a decrease in the population of white blood cells is observed in the sample from the patient after administration of the anti-IL-33 antibody or antigen binding fragment thereof as compared to that of the sample from the patient before administration of the anti-IL-33 antibody or antigen binding fragment thereof. In an embodiment of any of the foregoing methods, the level of white blood cells is measured 1 week or less after administration of the anti-IL-33 antibody or antigen binding fragment thereof. Following the selection of a patient according to any of the foregoing, the method can further include treating such patient with the inventive anti-IL-33 antibody or antigen binding fragment thereof.

### Anti-IL-33 Antibody or Antigen-Binding Fragment

Any of the foregoing methods are not limited to the use of any particular anti-IL-33 antibody or antibody fragment, provided the antibody or antibody fragment has an effect that is sufficiently rapid and persistent to allow for a therapeutic effect within the dosing parameters described herein. In an embodiment, the anti-IL-33 antibody or antigen binding fragment thereof of the inventive method binds to, and neutralizes, IL-33, thereby inhibiting IL-33 activity. The terms “inhibit” or “neutralize,” as used herein with respect to the activity of an anti-IL-33 antibody or antigen binding fragment, refer to the ability to substantially antagonize, prohibit, prevent, restrain, slow, disrupt, alter, eliminate, stop, or reverse the progression or severity of, for example, the biological activity of IL-33, or a disease or condition associated with IL-33, for example, atopic dermatitis. The inventive method preferably inhibits or neutralizes the activity of IL-33 by at least about 20%, about 30%, about 40%, about 50%, about 60%, about 70%, about 80%, about 90%, about 95%, about 100%, or a range defined by any two of the foregoing values.

The anti-IL-33 antibody or antigen-binding fragment suitable for use in the inventive method can be a whole antibody or an antibody fragment. The terms “fragment of an antibody,” “antibody fragment,” and “functional fragment of an antibody” are used interchangeably herein to mean one or more fragments of an antibody that retain the ability to specifically bind to an antigen (see, generally, Holliger et al., Nat. Biotech., 23(9): 1126-1129 (2005)). The anti-IL-33 antibody can contain any anti-IL-33 antibody fragment. The antibody fragment desirably comprises, for example, one or more CDRs, the variable region (or portions thereof), the constant region (or portions thereof), or combinations thereof. Examples of antibody fragments include, but are not limited to, (i) a Fab fragment, which is a monovalent fragment consisting of the V_{L}, V_{H}, C_{L}, and CH₁ domains, (ii) a F(ab′)2 fragment, which is a bivalent fragment comprising two Fab fragments linked by a disulfide bridge at the hinge region, (iii) a Fv fragment consisting of the V_{L} and V_{H} domains of a single arm of an antibody, (iv) a Fab′ fragment, which results from breaking the disulfide bridge of an F(ab′)₂ fragment using mild reducing conditions, (v) a disulfide-stabilized Fv fragment (dsFv), and (vi) a domain antibody (dAb), which is an antibody single variable region domain (V_{H} or V_{L}) polypeptide that specifically binds antigen.

The anti-IL-33 antibody or, in some embodiments, the antigen-binding fragment, can comprises a heavy chain constant region (Fₑ) of any suitable class. Preferably, the antibody or antibody fragment comprises a heavy chain constant region that is based upon wild-type IgG1, IgG2, or IgG4 antibodies, or variants thereof. In some embodiments, anti-IL-33 antibody or antigen-binding fragment comprises an Fc region engineered to reduce or eliminate effector functions of the antibody. Engineered Fc regions with reduced or abrogated effector function are known in the art and commercially available, as are techniques for engineering Fc regions to reduce or eliminate effector function, any of which can be used in conjunction with the invention.

The anti-IL-33 antibody or antigen-binding fragment also can be a single chain antibody fragment. Examples of single chain antibody fragments include, but are not limited to, (i) a single chain Fv (scFv), which is a monovalent molecule consisting of the two domains of the Fv fragment (i.e., V_{L} and V_{H}) joined by a synthetic linker which enables the two domains to be synthesized as a single polypeptide chain (see, e.g., Bird et al., Science, 242: 423-426 (1988); Huston et al., Proc. Natl. Acad. Sci. USA, 85: 5879-5883 (1988); and Osbourn et al., Nat. Biotechnol., 16: 778 (1998)) and (ii) a diabody, which is a dimer of polypeptide chains, wherein each polypeptide chain comprises a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a dimeric molecule having two functional antigen binding sites. Antibody fragments are known in the art and are described in more detail in, e.g., U.S. Patent Application Publication 2009/0093024 A1.

The anti-IL-33 antibody or antigen-binding fragment also can be an intrabody or fragment thereof. An intrabody is an antibody which is expressed and which functions intracellularly. Intrabodies typically lack disulfide bonds and are capable of modulating the expression or activity of target genes through their specific binding activity. Intrabodies include single domain fragments such as isolated V_{H} and V_{L} domains and scFvs. An intrabody can include sub-cellular trafficking signals attached to the N or C terminus of the intrabody to allow expression at high concentrations in the sub-cellular compartments where a target protein is located. Upon interaction with a target gene, an intrabody modulates target protein function and/or achieves phenotypic/functional knockout by mechanisms such as accelerating target protein degradation and sequestering the target protein in a non-physiological sub-cellular compartment. Other mechanisms of intrabody-mediated gene inactivation can depend on the epitope to which the intrabody is directed, such as binding to the catalytic site on a target protein or to epitopes that are involved in protein-protein, protein-DNA, or protein-RNA interactions.

The anti-IL-33 antibody or antigen-binding fragment also can be an antibody conjugate. In this respect, the anti-IL-33 antibody or antigen-binding fragment can be a conjugate of (1) an antibody, an alternative scaffold, or fragments thereof, and (2) a protein or non-protein moiety comprising the anti-IL-33 antibody or antigen-binding fragment. For example, the anti-IL-33 antibody or antigen-binding fragment can be all or part of an antibody conjugated to a peptide, a fluorescent molecule, or a chemotherapeutic agent.

The anti-IL-33 antibody or antigen-binding fragment can be, or can be obtained from, a human antibody, a non-human antibody, or a chimeric antibody. By “chimeric” is meant an antibody or fragment thereof comprising both human and non-human regions. Preferably, the anti-IL-33 antibody or antigen-binding fragment is a humanized antibody. A “humanized” antibody is a monoclonal antibody comprising a human antibody scaffold and at least one CDR obtained or derived from a non-human antibody. Non-human antibodies include antibodies isolated from any non-human animal, such as, for example, a rodent (e.g., a mouse or rat). A humanized antibody can comprise, one, two, or three CDRs obtained or derived from a non-human antibody. In one embodiment of the invention, CDRH3 of the inventive anti-IL-33 antibody or antigen-binding fragment may be obtained or derived from a mouse monoclonal antibody, while the remaining variable regions and constant region of the inventive anti-IL-33 antibody or antigen-binding fragment may be obtained or derived from a human monoclonal antibody.

A human antibody, a non-human antibody, a chimeric antibody, or a humanized antibody can be obtained by any means, including via in vitro sources (e.g., a hybridoma or a cell line producing an antibody recombinantly) and in vivo sources (e.g., rodents). Methods for generating antibodies are known in the art and are described in, for example, Köhler and Milstein, Eur. J. Immunol., 5: 511-519 (1976); Harlow and Lane (eds.), Antibodies: A Laboratory Manual, CSH Press (1988); and Janeway et al. (eds.), Immunobiology, 5th Ed., Garland Publishing, New York, N.Y. (2001)). In certain embodiments, a human antibody or a chimeric antibody can be generated using a transgenic animal (e.g., a mouse) wherein one or more endogenous immunoglobulin genes are replaced with one or more human immunoglobulin genes. Examples of transgenic mice wherein endogenous antibody genes are effectively replaced with human antibody genes include, but are not limited to, the Medarex HUMAB-MOUSE™, the Kirin TC MOUSE™, and the Kyowa Kirin KM-MOUSE™ (see, e.g., Lonberg, Nat. Biotechnol., 23(9): 1117-25 (2005), and Lonberg, Handb. Exp. Pharmacol., 181: 69-97 (2008)). A humanized antibody can be generated using any suitable method known in the art (see, e.g., An, Z. (ed.), Therapeutic Monoclonal Antibodies: From Bench to Clinic, John Wiley & Sons, Inc., Hoboken, N.J. (2009)), including, e.g., grafting of non-human CDRs onto a human antibody scaffold (see, e.g., Kashmiri et al., Methods, 36(1): 25-34 (2005); and Hou et al., J. Biochem., 144(1): 115-120 (2008)). In one embodiment, a humanized antibody can be produced using the methods described in, e.g., U.S. Patent Application Publication 2011/0287485 A1.

In one embodiment, a CDR (e.g., CDR1, CDR2, or CDR3) or a variable region of the immunoglobulin heavy chain polypeptide and/or the immunoglobulin light chain polypeptide of the anti-IL-33 antibody or antigen-binding fragment suitable for the inventive method can be transplanted (i.e., grafted) into another molecule, such as an antibody or non-antibody polypeptide, using either protein chemistry or recombinant DNA technology. In this regard, the invention provides an anti-IL-33 antibody or antigen-binding fragment comprising at least one CDR of an immunoglobulin heavy chain and/or light chain polypeptide as described herein. The anti-IL-33 antibody or antigen-binding fragment can comprise one, two, or three CDRs of an immunoglobulin heavy chain and/or light chain variable region as described herein.

The anti-IL-33 antibody or antigen binding fragment of the invention may be comprised of an isolated immunoglobulin heavy chain polypeptide and/or an isolated immunoglobulin light chain polypeptide, or a fragment (e.g., antigen-binding fragment) thereof. The term “antibody” or “immunoglobulin” as used herein, refers to a protein that is found in blood or other bodily fluids of vertebrates, which is used by the immune system to identify and neutralize foreign objects, such as bacteria and viruses. The polypeptide is “isolated” in that it is removed from its natural environment. In a preferred embodiment, an antibody or immunoglobulin is a protein that comprises at least one complementarity determining region (CDR). The CDRs form the “hypervariable region” of an antibody, which is responsible for antigen binding. A whole immunoglobulin typically consists of four polypeptides: two identical copies of a heavy (H) chain polypeptide and two identical copies of a light (L) chain polypeptide. Each of the heavy chains contains one N-terminal variable (V_{H}) region and three C-terminal constant (C_{H}1, C_{H}2, and C_{H}3) regions, and each light chain contains one N-terminal variable (V_{L}) region and one C-terminal constant (C_{L}) region. The light chains of antibodies can be assigned to one of two distinct types, either kappa (κ) or lambda (λ), based upon the amino acid sequences of their constant domains. In a typical immunoglobulin, each light chain is linked to a heavy chain by disulfide bonds, and the two heavy chains are linked to each other by disulfide bonds. The light chain variable region is aligned with the variable region of the heavy chain, and the light chain constant region is aligned with the first constant region of the heavy chain. The remaining constant regions of the heavy chains are aligned with each other.

The variable regions of each pair of light and heavy chains form the antigen binding site of an antibody. The V_{H} and V_{L} regions have the same general structure, with each region comprising four framework (FW or FR) regions. The term “framework region,” as used herein, refers to the relatively conserved amino acid sequences within the variable region which are located between the hypervariable or complementary determining regions (CDRs). There are four framework regions in each variable domain, which are designated FR1, FR2, FR3, and FR4. The framework regions form the 13 sheets that provide the structural framework of the variable region (see, e.g., C. A. Janeway et al. (eds.), Immunobiology, 5th Ed., Garland Publishing, New York, N.Y. (2001)).

The framework regions are connected by three complementarity determining regions (CDRs). The three CDRs, known as CDR1, CDR2, and CDR3, form the “hypervariable region” of an antibody, which is responsible for antigen binding. The CDRs form loops connecting, and in some cases comprising part of, the beta-sheet structure formed by the framework regions. While the constant regions of the light and heavy chains are not directly involved in binding of the antibody to an antigen, the constant regions can influence the orientation of the variable regions. The constant regions also exhibit various effector functions, such as participation in antibody-dependent complement-mediated lysis or antibody-dependent cellular toxicity via interactions with effector molecules and cells.

Examples of suitable anti-IL-33 antibodies or antigen binding fragments for use in the method are any of those described in WO 2015/106080 A2, the entire disclosure of which is specifically incorporated herein by reference. In an embodiment, the anti-IL-33 antibody or antigen-binding fragment thereof may comprise: a heavy chain variable region comprising a complementary determining region (CDR) 1 domain (CDRL1) comprising the amino acid sequence of SEQ ID NO: 1; a CDRL2 domain comprising the amino acid sequence of SEQ ID NO:2; and a CDRL3 domain comprising the amino acid sequence of SEQ ID NO: 3, and a light chain variable region comprising a CDRH1 domain comprising the amino acid sequence of SEQ ID NO:4; a CDRH2 domain comprising the amino acid sequence of SEQ ID NO:5; and a CDRH3 domain comprising the amino acid sequence of SEQ ID NO: 6. In another embodiments, the anti-IL-33 antibody or antibody fragment may comprise a heavy chain variable region of SEQ ID NO: 7 and/or a light chain variable region of SEQ ID NO: 8.

In another embodiment, the anti-IL-33 antibody or antigen-binding fragment thereof is an antibody or antibody fragment that competes with an antibody comprising a heavy chain variable region of SEQ ID NO: 7 and a light chain variable region of SEQ ID NO: 8 for binding to IL-33. In yet another embodiment, the anti-IL-33 antibody or antigen-binding fragment thereof is an antibody or antibody fragment that competes with ST2 for binding to IL-33.

To express the antibodies, or antibody portions of the invention, DNAs encoding partial or full-length light and heavy chains, obtained as described above, are inserted into expression vectors such that the genes are operatively linked to transcriptional and translational control sequences. In this context, the term “operatively linked” is intended to mean that an antibody gene is ligated into a vector such that transcriptional and translational control sequences within the vector serve their intended function of regulating the transcription and translation of the antibody gene. The expression vector and expression control sequences are chosen to be compatible with the expression host cell used. The antibody light chain gene and the antibody heavy chain gene can be inserted into separate vector or, more typically, both genes are inserted into the same expression vector. The antibody genes are inserted into the expression vector by standard methods (e.g., ligation of complementary restriction sites on the antibody gene fragment and vector, or blunt end ligation if no restriction sites are present). Prior to insertion of the anti-IL-33-related light or heavy chain sequences, the expression vector may already carry antibody constant region sequences. For example, one approach to converting the anti-IL-33-related VH and VL sequences to full-length antibody genes is to insert them into expression vectors already encoding heavy chain constant and light chain constant regions, respectively, such that the VH segment is operatively linked to the CH segment(s) within the vector and the VL segment is operatively linked to the CL segment within the vector. Alternatively or additionally, the recombinant expression vector can encode a signal peptide that facilitates secretion of the antibody chain from a host cell. The antibody chain gene can be cloned into the vector such that the signal peptide is linked in-frame to the amino terminus of the antibody chain gene. The signal peptide can be an immunoglobulin signal peptide or a heterologous signal peptide (i.e., a signal peptide from a non-immunoglobulin protein).

In addition to the antibody chain genes, the recombinant expression vectors of the invention carry regulatory sequences that control the expression of the antibody chain genes in a host cell. The term “regulatory sequence” is intended to include promoters, enhancers and other expression control elements (e.g., polyadenylation signals) that control the transcription or translation of the antibody chain genes. Such regulatory sequences are described, for example, in Goeddel; Gene Expression Technology: Methods in Enzymology 185, Academic Press, San Diego, Calif. (1990). It will be appreciated by those skilled in the art that the design of the expression vector, including the selection of regulatory sequences may depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see e.g., U.S. Pat. No. 5,168,062 by Stinski, U.S. Pat. No. 4,510,245 by Bell et al. and U.S. Pat. No. 4,968,615 by Schaffner et al.

In addition to the antibody chain genes and regulatory sequences, the recombinant expression vectors which may be suitable for use in the inventive methods may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

For expression of the light and heavy chains, the expression vector(s) encoding the heavy and light chains is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is theoretically possible to express the anti-IL-33 antibody or antigen-binding fragment of the invention in either prokaryotic or eukaryotic host cells, expression of antibodies in eukaryotic cells, and most preferably mammalian host cells, is the most preferred because such eukaryotic cells, and in particular mammalian cells, are more likely than prokaryotic cells to assemble and secrete a properly folded and immunologically active antibody. Prokaryotic expression of antibody genes has been reported to be ineffective for production of high yields of active antibody (Boss, M. A. and Wood, C. R. (1985) Immunology Today 6:12-13).

Preferred mammalian host cells for expressing the antibodies of the invention include Chinese Hamster Ovary (CHO cells) (including dhfr⁻ CHO cells, described in Urlaub and Chasin, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NSO myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce portions of intact antibodies, such as Fab fragments or scFv molecules. It will be understood that variations on the above procedure are within the scope of the present invention. For example, it may be desirable to transfect a host cell with DNA encoding either the light chain or the heavy chain (but not both) of an antibody of this invention. Recombinant DNA technology may also be used to remove some or all of the DNA encoding either or both of the light and heavy chains that is not necessary for binding to IL-33. The molecules expressed from such truncated DNA molecules are also encompassed by the antibodies of the invention. In addition, bifunctional antibodies may be produced in which one heavy and one light chain are an antibody of the invention and the other heavy and light chain are specific for an antigen other than IL-33 by crosslinking an antibody of the invention to a second antibody by standard chemical crosslinking methods.

In a preferred system for recombinant expression of an antibody, or antigen-binding portion thereof, of the invention, a recombinant expression vector encoding both the antibody heavy chain and the antibody light chain is introduced into dhfr⁻CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are culture to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium.

### Pharmaceutical Compositions

The anti-IL-33 antibody or antigen binding fragment of the inventive method can be formulated into a composition, such as a pharmaceutical composition, for administration to a patient. Typically, the pharmaceutical composition comprises the antibody or antigen binding fragment thereof of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible and are suitable for administration to a subject for the methods described herein. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

As will be appreciated by the skilled artisan, the route and/or mode of administration will vary depending upon the desired results. The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In a preferred embodiment, the anti-IL-33 antibody or antigen binding fragment thereof is administered by intravenous infusion or injection. In another preferred embodiment, the antibody is administered by intramuscular injection. In a particularly preferred embodiment, the antibody is administered by subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antigen binding fragment) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyethylene glycol (PEG), polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

The terms “treat” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of atopic dermatitis. Also, for purposes herein, “prevention” can encompass delaying the onset of the disease, or a symptom or condition thereof.

The following examples further illustrate the invention but, of course, should not be construed as in any way limiting its scope.

### Example 1

This example demonstrates the effect of anti-IL-33 (ANB020) on atopic dermatitis.

ANB020 is a novel, humanized anti-human IL-33 monoclonal antibody which shows high affinity binding to human IL-33 with a K_{d} of approximately 1 pM. ANB020 provides a potent IL-33 neutralizing activity with an IC₅₀ of approximately 1.5 nM.

Twelve patients with moderate to severe atopic dermatitis were enrolled in the study. The atopic dermatitis of all 12 patients was inadequately controlled by topical corticosteroids, and seven were previously treated with systemic non-biologic anti-inflammatory therapy, prior to the screening washout of this trial. The baseline characteristics of the 12 patients are shown in Table 1 below.

TABLE 1

| Characteristic | |
|---|---|
| Age (years) | 40.4 ± 13.5 |
| Male, number (%) | 11 (91.7%) |
| Caucasian race, number (%) | 12 (100%) |
| Body-Mass Index | 26.14 ± 4.145 |
| EASI, score | 32.25 ± 10.89 |
| IGA, 0-5scale | 4 ± 0.74 |
| SCORAD, score | 64.79 ± 12.02 |
| Pruritus, 5-D score | 19.1 ± 4.85 |
| DLQI, score | 12.92 ± 6.54 |
| Eosinophils, per microliter blood | 588 ± 468 |

Pre-specified pharmacodynamic endpoints were changes in skin suction blister contents, and differential white blood cell counts. Main clinical endpoints were scores for Eczema Area and Severity Index (EASI), Investigator's Global Assessment (IGA), Severity scoring of atopic dermatitis (SCORAD), DLQI, and 5D Pruritus Score; and patient diary data.

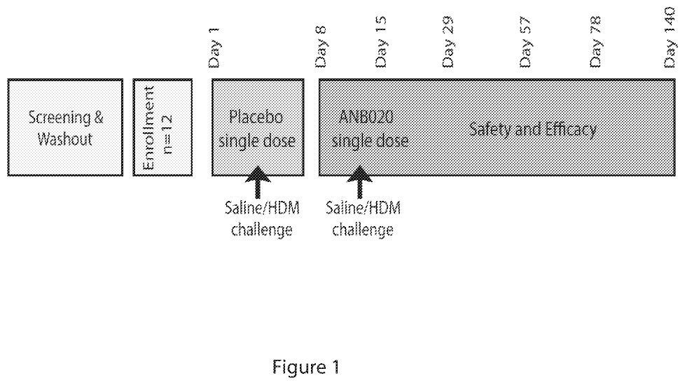

On day 1 of the study, each patient was administered a placebo (saline) and a baseline EASI score was recorded for each patient at 24 hours after placebo injection. On day 4, each patient was administered an HDM skin challenge via subcutaneous injection of HDM in saline. On day 8 of the study, each patient was administered a single 300 mg dose (iv) of ANB020. Day 8 of the study is, therefore, day 0 in the post-ANB020 administration schedule. On day 11 of the study, patients again received an HDM skin challenge. EASI scores were measured on days 15, 29, 57, 78, 113, and 140 post-ANB020 administration (FIG. 1).

Table 2 presents the average EASI scores following the single ANB020 dose as a percentage of the baseline EASI score. As noted, a rapid response was achieved in all patients on or before day 57.

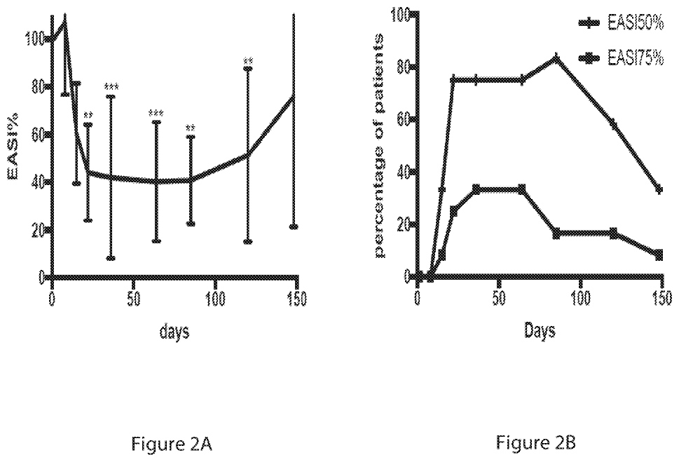

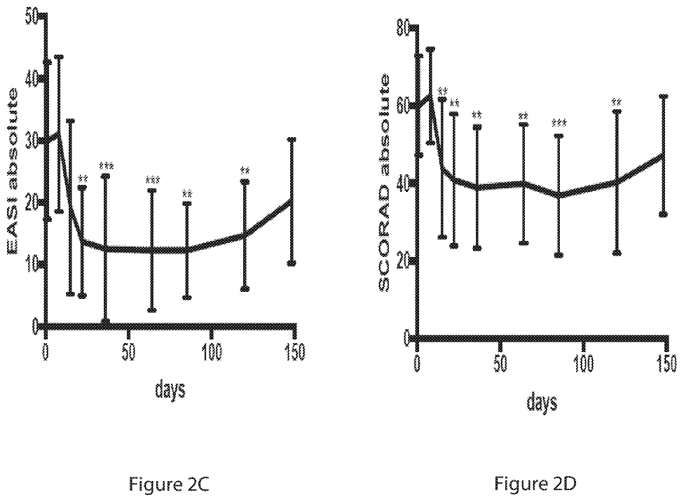

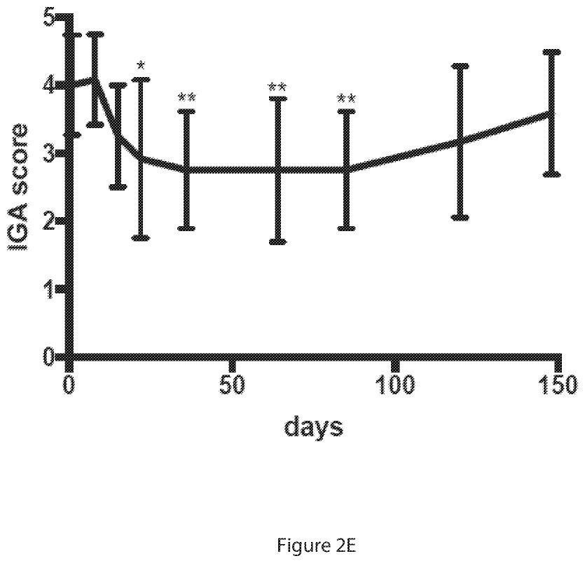

The data shows that by 29 days after ANB020 administration, a marked improvement in EASI score was observed with 83% of patients showing EASI50 and 33% showing EASI75, with a mean EASI improvement of 59% (P<0.001; FIGS. 2A-2C). Even 15 days after ANB020, a significant improvement was observed (mean 58% EASI decrease, P<0.01) compared to baseline. Responses were sustained with 75% of patients showing EASI50 and 42% of patients showing EASI75 at day 57 post-administration, with a mean EASI improvement of 63% (P<0.001). Responses were further sustained up to 140 days post-administration with 42% of patients showing EASI50 and 25% of patients showing EASI75 (Table 2).

These results demonstrate that ANB020 induced a rapid clinical benefit within two weeks of dosing, reaches clinical efficacy threshold (EASI50) in all patients, and will persist for at least about four to four and a half months after a single dose. No severe adverse events were reported and ANB020 was generally well tolerated amongst all patients. Thus, dosing with ANB020 every 4, 8, 12, 16, or 20 weeks is likely to maintain EASI efficacy amongst atopic dermatitis patients.

TABLE 2
Average EASI Score % Reduction of patients administered a single dose of ANB020.

| | | Day 1 | | | | | | |
|---|---|---|---|---|---|---|---|---|
| | Day −21 | ANB020 | Day | Day | Day | Day | Day | Day |
| Patient | Baseline | Dosing | 15 | 29 | 57 | 78 | 113 | 140 |
| Average % Easy | 0% | 4% | 58% | 61% | 62% | 62% | 55% | 45% |
| Score Reduction | | | | | | | | |
| EASI50 Response | 0 | 0 | 9 of 12 | 10 of 12 | 9 of 12 | 9 of 12 | 8 of 12 | 5 of 12 |
| | | | (75%) | (83%) | (75%) | (75%) | (67%) | (42%) |
| EASI75 Response | 0 | 0 | 3 of 12 | 4 of 12 | 5 of 12 | 2 of 12 | 2 of 12 | 3 of 12 |
| | | | (25%) | (33%) | (42%) | (17%) | (17%) | (25%) |

### Example 2

This example demonstrates the effect of AN020 on pruritis in moderate-to-severe atopic dermatitis patients.

Pruritus in the patients enrolled in the study described in Example 1 was assessed according to the 5-D pruritis itch scale (see Elman et al., Br J Dermatol., 162(3):587-93 (2010)) during screening and on days 1, 15, 29, 57, 78, 113, and 140 of the study. The results are presented in Table 3. An average of the scores show that, by day 36 of the study (29 days post-ANB020 administration, pruritis was reduced to about 32% of the average baseline score. At day 57 post-ANB020 administration, average pruritus reduction was 21% relative to baseline. At day 140 post-ANB020 administration, average pruritus reduction was still 21% relative to baseline.

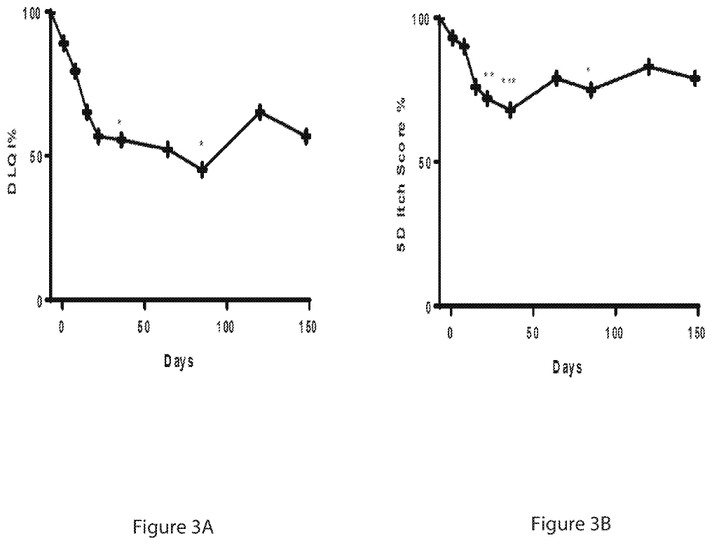

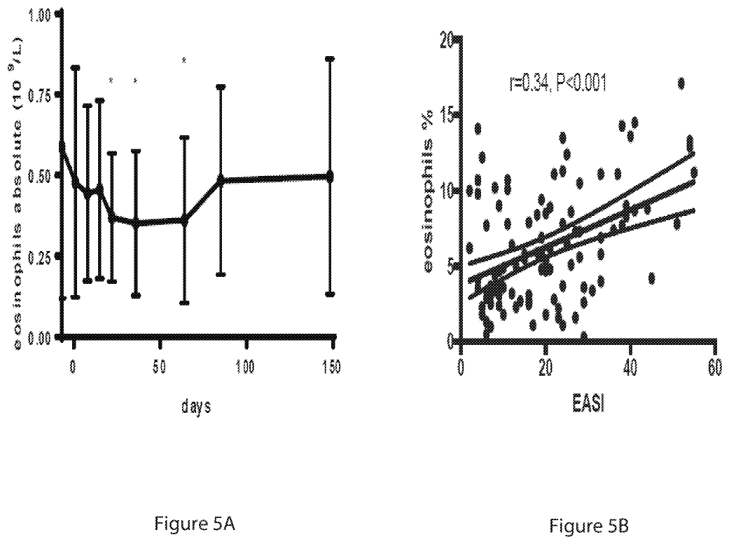

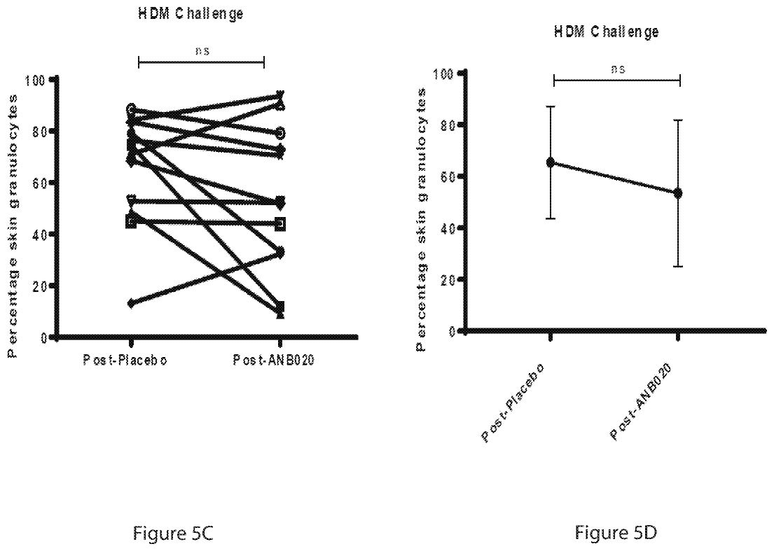

Additional efficacy data was measured in all twelve patients on days 15, 29, 57, 78, 113, and 140 post-ANB020 administration (Table 3). Objective clinical outcomes were associated with significant improvement in Dermatology Life Quality Index (DLQI) (P<0.05) and 5D itch scores (P<0.01). There were significant improvements in SCORAD at day 29 with 40% reduction (P<0.01; FIG. 2D). Three (25%) patients reached an IGA of 0/1 during the study (FIG. 2E). The objective clinical outcomes were associated with significant improvement in DLQI (P<0.05; FIG. 3A) and 5D itch scores (P<0.001; FIG. 3B).

Blood eosinophil reduction, which is a biomarker illustrative of ANB020's mechanistic breadth, was observed (Table 4). The EASI score at study entry showed a significant correlation with peripheral eosinophil percentage (r=0.623, P<0.0001). There was a significant reduction in peripheral eosinophil absolute counts at day 29 post-administration (mean 40% reduction, P<0.05). Peripheral eosinophil percentage counts at day 29 post-administration were also significantly reduced (mean 40% reduction, P<0.05, FIG. 5A) and eosinophil percentage count throughout the study correlated with EASI score (r=0.3419, P<0.001, FIG. 5B).

ANB020 inhibition of IFNγ production by whole blood in response to IL-33/IL-12 correlated with EASI reduction (r=0.34, P<0.05) mirroring durability of clinical activity (Table 4). Furthermore, ANB020 significantly inhibited granulocyte infiltration into the skin (P<0.05), and group 2 innate lymphoid cell type 2 cytokine response to IL-33 (P<0.0001).

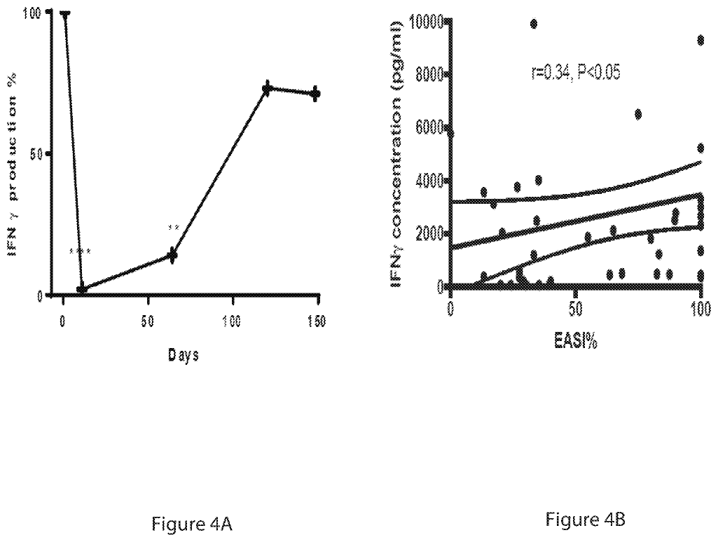

The in vivo pharmacodynamic (PD) effects in the setting of disease was investigated using whole blood stimulation with IL-33/IL-12, and IFNγ production was measured by ELISA (FIG. 4A). Whole blood was incubated at 37° C. for 16 hours with IL-12 and IL-33 at 30 and 50 ng/mL, respectively. The inhibition of IFNγ production was rapid and dramatic, and was observed to extend beyond 57 days in all patients and to beyond 120 days in some individuals (FIG. 4A). Whether the IFNγ response to IL-33/IL-12 was related to the change in disease severity was investigated, and a significant positive correlation with EASI % (r=0.3453, P<0.05, FIG. 4B) was observed.

TABLE 3
5-D Itch, SCORAD, DLQI, and IGA Score Data relative to baseline upon enrollment
at Day −21 for patients administered a single dose of ANB020.

| | | Day 1 | | | | | | |
|---|---|---|---|---|---|---|---|---|
| | Day −21 | ANB020 | Day | Day | Day | Day | Day | Day |
| Patient | Baseline | Dosing | 15 | 29 | 57 | 78 | 113 | 140 |
| Average % 5-D Pruritis | 0% | 10% | 28% | 32% | 21% | 25% | 17% | 24% |
| Score Reduction | | | | | | | | |
| Average % SCORAD | 0% | 3% | 37% | 40% | 38% | 40% | 38% | 32% |
| Reduction | | | | | | | | |
| Average % DLQI | 0% | 21% | 43% | 45% | 48% | 55% | 35% | 43% |
| Reduction | | | | | | | | |

TABLE 4
Biomarker data at specific timepoints relative to baseline upon enrollment
at Day −21 for patients administered a single dose of ANB020.

| | | Day 1-4* | | | | | | |
|---|---|---|---|---|---|---|---|---|
| | Day −21 | ANB020 | Day | Day | Day | Day | Day | Day |
| Timepoint | Baseline | Dosing | 15 | 29 | 57 | 78 | 113 | 140 |
| % Blood | 0% | 25% | 37% | 40% | 39% | 18% | Not | 16% |
| Eosinophil | | | | | | | measured | |
| Reduction | | | | | | | | |
| % Ex Vivo | 0% | 98% | Not | Not | 86% | Not | 27% | 29% |
| I1-33 | | | measured | measured | | measured | | |
| Mediated | | | | | | | | |
| IFN-γ | | | | | | | | |
| Release | | | | | | | | |
| Reduction | | | | | | | | |
| % Patients | 0% | 0% | 75% | 83% | 75% | 75% | 67% | 42% |
| Achieving | | | | | | | | |
| EASI-50 | | | | | | | | |
| % Patients | 0% | 0% | 25% | 33% | 42% | 17% | 17% | 25% |
| Achieving | | | | | | | | |
| EASI-75 | | | | | | | | |

*6-72 hours post ANB020 dose.

### Example 3

This example compares the efficacy of single dose ANB020 to weekly dosing of Dupilumab, an anti-IL-4Ra antibody that received “breakthrough” designation from the FDA for treatment of moderate-to-severe atopic dermatitis. The comparison is presented in Table 4 below. Dupilumab EASI and pruritus data is taken from Beck et al., N. Engl. J. Med., 10; 371 (2), 130-139 (2014)), while safety data was obtained from the FDA approved product label for Dupilumab (Dupixent®).

TABLE 4
Comparison of ANB020 to Dupilumab

| | | ANB020 | Dupilumab | |
|---|---|---|---|---|
| | | (single dose 300 | (weekly 300 mg | Dupilumab |
| | | mg IV at day 29 | SC) at day 29 | (weekly 300 mg |
| | Relevant | post admin; | post admin; | SC for 12 weeks |
| | Threshold | n = 12) | n = 55 | n = 55) |
| Efficacy | Patients | 83% (10 of 12) | 69% drug vs 29% | 85% drug vs 35% |
| Endpoint | Achieving | | placebo | placebo |
| | EASI-50 | | | |
| | Response | | | |
| | Patients | 33% (4 of 12) | 29% drug vs | 62% drug vs 15% |
| | Achieving | | 6% placebo | placebo |
| | EASI-75 | | | |
| | Response | | | |
| | 5-D Score | 38% reduction | 41% reduction | 56% reduction |
| | Reduction to | | drug vs 19% | drug vs. 15% |
| | Baseline | | placebo | placebo |

| | | |
|---|---|---|
| Safety | No SAEs to date | 10-20% conjunctivitis |

### Example 4

This example illustrates the effect of ANB020 on the leukocyte population of HDM-challenged blisters in atopic dermatitis patients.

The skin of each patient enrolled in the study described in Example 1 was challenged contra-laterally with an injection of saline or HDM four days following initial administration of the placebo. Fluid from blisters formed at the injection sites was extracted and analyzed one day later (5-days post placebo administration). A single systemic (iv) 300 mg dose of ANB020 was administered on day 8 of the study, and patients again underwent saline and HDM skin challenge (contra-laterally) on day 11 (day 3 post-ANB020 administration). Fluid from blisters formed at the injection sites was extracted and analyzed on day 12 (day 5 post-ANB020 administration). Fluids were analyzed by fluorescence-activated cell sorting (FACS) to determine the populations of lymphocytes, granulocytes, and monocytes per microliter of blister fluid. HDM challenged blisters generally had a greater leukocyte population, with a significantly increased granulocyte population, relative to the saline challenged blisters.

Specifically, granulocyte infiltration into the skin was reduced in response to skin suction blister formation with saline challenge after ANB020 compared to that observed after placebo (mean 37% reduction, P=0.05, FIG. 5C). Granulocyte infiltration into the skin showed a reduction trend in response to skin suction blister formation with HDM challenge after ANB020 compared to that observed after placebo, but this did not reach statistical significance (mean 30% reduction, P=0.13, FIG. 5D).

Cell populations from the saline- and HDM-challenged blisters were analyzed as a % of total leukocytes both before ANB020 administration and after ANB020 administration. Before administration of ANB020, the HDM challenged blisters contained a significantly greater % of granulocytes relative to saline challenged blisters, while the lymphocyte population was reduced in HDM blisters as compared to saline blisters, and monocytes were relatively unchanged between saline and HDM challenged blisters. After ANB020 administration, the % of granulocytes in HDM challenged blisters was reduced to approximately placebo saline level. The lymphocyte levels increased as a % of total leukocytes while the monocytes remained relatively unchanged.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating atopic dermatitis in a patient comprising administering to the patient a dose of an anti-IL-33 antibody or antigen binding fragment thereof not more than once every two weeks.

2. (canceled)

3. The method of claim 1, wherein the method comprises administering to the patient a dose of an anti-IL-33 antibody or antigen binding fragment thereof not more than once every four weeks.

4. (canceled)

5. The method of claim 1, wherein the method comprises administering to the patient a dose of an anti-IL-33 antibody or antigen binding fragment thereof not more than once every eight weeks.

6. (canceled)

7. The method of claim 1, wherein the method comprises administering to the patient a dose of an anti-IL-33 antibody or antigen binding fragment thereof not more than once every 12 weeks.

8. The method of claim 1, wherein the method comprises administering to the patient a dose of an anti-IL-33 antibody or antigen binding fragment thereof not more than once every 16 weeks.

9. (canceled)

10. The method of claim 1, wherein the dose is about 40-600 mg.

11. The method of claim 1, wherein the dose is about 40-300 mg.

12. The method of claim 1, wherein the method comprises administration of a single loading dose followed by a maintenance dose not more than once every two weeks, wherein the loading dose is an amount equal to 1.5× to 4× the maintenance dose.

13. (canceled)

14. The method of claim 12, wherein the maintenance dose is administered not more than once every four weeks.

15. The method of claim 12, wherein the maintenance dose is administered not more than once every eight weeks.

16. The method of claim 12, wherein the maintenance dose is administered not more than once every twelve weeks.

17. The method of any of claim 1, wherein the patient achieves 50% improvement relative to the patient's baseline EASI score within four weeks.

18. The method of any of claim 1, wherein the method applied to a population of at least 12 patients achieves a 50% improvement in at least 50% of the patients relative to their baseline EASI score within four weeks.

19. The method of any of claim 1, wherein the method applied to a population of at least 12 patients achieves a 50% improvement in at least 75% of the patients relative to their baseline EASI score within four weeks.

20. (canceled)

21. The method of claim 1, wherein the anti-IL-33 antibody or antibody fragment thereof is administered subcutaneously.

22. A method of selecting a patient with atopic dermatitis for treatment with an anti-IL-33 antibody or antigen binding fragment thereof, the method comprising
(a) administering an anti-IL-33 antibody or antigen binding fragment thereof to the patient; and
(b) comparing the level of white blood cells in the patient's blood or in a disease lesion on the patient's skin after administration of the anti-IL-33 antibody or antigen binding fragment thereof to the level of white blood cells in the patient's blood or in a disease lesion on the patient's skin before administration of the anti-IL-33 antibody or antigen binding fragment thereof;
and selecting the patient for treatment when a decrease in the population of white blood cells is observed after administration of the anti-IL-33 antibody or antigen binding fragment thereof;
or
comparing the level of itching or pruritus exhibited by the patient after administration of the anti-IL-33 antibody or antigen binding fragment thereof to the level of itching or pruritus exhibited by the patient before administration of the anti-IL-33 antibody or antigen binding fragment thereof;
and selecting the patient for treatment when a decrease in the itching or pruritus is observed after administration of the anti-IL-33 antibody or antigen binding fragment thereof as compared to before administration of the anti-IL-33 antibody or antigen binding fragment thereof.

23. (canceled)

24. The method of claim 22, further comprising treating the selected patient with an anti-IL-33 antibody.

25. The method of claim 22, wherein the level of white blood cells in a disease lesion on the patient's skin is measured 1 week or less after administration of the anti-IL-33 antibody or antigen binding fragment thereof.

26. The method of claim 1, wherein the anti-IL-33 antibody or antigen-binding fragment thereof comprises:
a heavy chain variable region comprising a complementary determining region (CDR) 1 domain (CDRL1) comprising the amino acid sequence of SEQ ID NO: 1; a CDRL2 domain comprising the amino acid sequence of SEQ ID NO:2; and a CDRL3 domain comprising the amino acid sequence of SEQ ID NO: 3, and
a light chain variable region comprising a CDRH1 domain comprising the amino acid sequence of SEQ ID NO:4; a CDRH2 domain comprising the amino acid sequence of SEQ ID NO:5; and a CDRH3 domain comprising the amino acid sequence of SEQ ID NO: 6

27. The method of claim 1, wherein the anti-IL-33 antibody comprises a heavy chain variable region comprising the amino acid sequence of SEQ ID NO: 7 and a light chain variable region comprising the amino acid sequence of SEQ ID NO: 8.

28.-56. (canceled)


SEQUENCE LISTING (8 sequences)

SEQ ID NO: 1 - protein, 10 aa, Artificial Sequence
  Synthetic Sequence
GYTFTSYWMH

SEQ ID NO: 2 - protein, 17 aa, Artificial Sequence
  Synthetic Sequence
TIYPRNSNTDYNQKFKA

SEQ ID NO: 3 - protein, 13 aa, Artificial Sequence
  Synthetic Sequence
PLYYYLTSPPTLF

SEQ ID NO: 4 - protein, 11 aa, Artificial Sequence
  Synthetic Sequence
KASQDVGTAVA

SEQ ID NO: 5 - protein, 7 aa, Artificial Sequence
  Synthetic Sequence
WASTRHT

SEQ ID NO: 6 - protein, 9 aa, Artificial Sequence
  Synthetic Sequence
QQAKTYPFT

SEQ ID NO: 7 - protein, 139 aa, Artificial Sequence
  Synthetic Sequence
MRAWIFFLLCLAGRALAQVQLMQSGAEVKKPGASVKVSCKASGYTFTSYWMHWVRQAPGQGLEWMGTIYPRNSNTDYNQKFKARVTMTRDTSTSTVYMELSSLRSEDTAVYYCARPLYYYLTSPPTLFWGQGTLVTVSS

SEQ ID NO: 8 - protein, 125 aa, Artificial Sequence
  Synthetic Sequence
MRAWIFFLLCLAGRALADIQLTQSPSFLSASVGDRVTITCKASQDVGTAVAWYQQKPGKAPKLLIYWASTRHTGVPSRFSGSGSGTEFTLTISSLQPEDFATYYCQQAKTYPFTFGSGTKLEIKR